(19) United States
(12) Patent Application Publication
(10) Pub. No.: US 2004/0259213 A1
(43) Pub. Date: Dec. 23, 2004

(54) Process of producing mannitol and homopolysaccharides

(76) Inventors: Gerritdina Hendrika Van Geel-Schutten, Driebergen (NL); Doede Jacob Binnema, Groningen (NL); Marc Jos Elise Cornelis Van Der Maarel, Haren (NL)

Correspondence Address: YOUNG & THOMPSON, 745 SOUTH 23RD STREET, 2ND FLOOR, ARLINGTON, VA, 22202, US

(21) Appl. No.: 10/484,714

(22) PCT Filed: Jul. 24, 2002
(86) PCT No.: PCT/NL02/00500

(30) Foreign Application Priority Data
Jul. 25, 2001 (EP) 01202842.9

(51) Int. Cl. C12P 19/04; C12P 7/18; C12N 1/20

(52) U.S. Cl. 435/101; 435/252.3; 435/252.9; 435/158

(57) ABSTRACT

The present invention relates to a process producing mannitol and one or more homopolysaccharides by bacteria. The bacteria according to the invention express mannitol-2-dehydrogenase activity and one or more sucrase activities. Preferably, the bacteria are lactic acid bacteria and more preferably the bacteria are selected from the genera Lactobacillus. Leuconostoc and Streptococcus. The invention also pertians to the use of said bacteria in the production of mannitol and one or more homopolysaccharides.

### FIELD OF THE INVENTION

The present invention is in the field of fermentation of bacteria and in particular of lactic acid bacteria The invention is particularly concerned with a process of producing mannitol and with the use of bacterial strains capable of producing this product.

### BACKGROUND OF THE INVENTION

Sugar alcohols, also called polyols, comprise a large group of compounds that are classified according to the number of hydroxyl groups (and carbon atoms) as tetritols, pentitols, hexitols and heptitols. Most of these compounds are water-soluble and crystalline with small optical rotations in water and have a slightly sweet to very sweet taste. Sugar alcohols are used in many fields, including foods, pharmaceuticals, cosmetics, textiles and polymers.

Of all the sugar alcohols sorbitol and mannitol have the greatest industrial significance with mannitol accounting for approximately 13% of the sugar alcohol market. Mannitol is widespread in nature. It is found in exudates of trees and shrubs such as the plane tree, manna ash and olive tree. It is also found in the fruit, leaves and other parts of various plants including pumpkin, hedge parsley, onions, celery, strawberries, cocoa bean, grasses, mistletoe, lilac and lichens. Furthermore, it can be found in marine algae, especially brown seaweed, the mycelia of several fungi and mushrooms.

Mannitol is used as a food additive in inter alia pressed mints, soft candy, cough drops, chewing gum, confections and frostings, jams and jellies. A concomitant advantage of the use of mannitol in food is that most oral bacteria cannot metabolize mannitol and thus does not promote tooth decay, and further that mannitol is slowly absorbed, resulting in a significantly reduced rise in blood glucose and insulin response. Furthermore, mannitol is added to bacteriological media and blood to protect it during storage and it can be used as a diuretic when administered intravenously. The main use of mannitol is however in pharmaceutical applications. It is used as a base in chewable, multilayer and press-coated tablets of vitamins, antacids, aspirin and other pharmaceuticals, because it provides a sweet taste, disintegrates smoothly and masks the unpleasant taste of drugs. Besides that, mannitol is a popular additive in pharmaceuticals because of its low hygroscopicity and its resistance to occlusion of water, making it an excellent additive especially for moisture-sensitive drugs.

Commercially, mannitol is mainly produced by hydrogenation of invert sugar. In alkaline media, glucose, fructose and mannose are interconverted. All of the mannose formed can then be reduced to mannitol. A disadvantage of this production method is that sorbitol is produced simultaneously and an isolation and purification step has to be introduced. Mannitol can also be produced by hydrogenation of starch hydrolysates in alkaline media in the presence of Raney nickel and it can also be obtained by extraction from seaweed.

Furthermore, it is known that mannitol can be produced by micro-organisms including fungi and bacteria For instance the lactic acid bacteria, designated Lactobacillus sp. Y-107 and Leuconostoc sp. Y-002 and isolated during fermentation of kimchi, a Korean fermented food product, were found to produce mannitol (see Yun, J. W. & Kim, D. H., The Journal of Fermentation and Bioengineering (1998) 85, 203-208). Both strains were capable of producing mannitol when grown on sucrose and fructose. Maximal mannitol production was obtained with fructose as a sole carbon source.

In recent years, lactic acid bacteria have received much attention because they are organisms having the GRAS (food-grade) status. They produce an abundant variety of exopolysaccharides (EPS's). These polysaccharides are thought to contribute to human health by acting as prebiotic substrates, nutraceuticals, cholesterol lowering agents or immunomodulants. Some lactic acid bacteria are able to synthesize homopoly-saccharides, i.e. glucans and fructans, using extracellular sucrase enzymes. Sucrases include fructansucrases, also called fructosyltransferases, such as inulosucrase (for example E.C. 2.4.1.9) and levansucrase (for example E.C. 2.4.1.10) synthesizing inulin and levan, respectively, and glucansucrases, also called glucosyltransferases, synthesizing various glucans.

The nomenclature of glucansucrases is rather confusing. The enzymes from Leuconostoc species are commonly called dextransucrases, whereas the streptococcal enzymes are referred to as glucosyltransferases. Other glucansucrases are for example alternansucrase (E.C. 2.4.1.140), amylosucrase (E.C. 2.4.2.4), a streptococcal glucosyl-transferase synthesizing mutan (E.C. 2.4.1.-) and a glucansucrase from Lactobacillus reuteri synthesizing a unique and highly branched glucan with α-(1-4) and α-(1-6) glucosidic bonds. Sucrose is a substrate for both glucansucrases and fructansucrases, whereas raffinose is a substrate for fructansucrases only. The sucrase enzymes synthesize glucans or fructans from sucrose, thereby releasing fructose of glucose, respectively. The sucrase enzymes are also capable of hydrolysing sucrose into glucose and fructose. For instance, the Lactobacillus reuteri strain LB 121 was found to produce both a glucan and a fructan when grown on sucrose as sole carbon source, but only a fructan when grown on raffinose as sole carbon source (van Geel-Schutten, G. H. et al., Appl. Microbiol. Biotechnol. (1998) 50, 697-703). According to another report, Lactobacillus reuteri strain LB 35-5, a spontaneous mutant of Lactobacillus reuteri strain LB 121, only produced a glucan when grown on sucrose as sole carbon source (van Geel-Schutten, G. H. et al., Appl. Environ. Microbiol. (1999) 65, 3008-3014).

### SUMMARY OF THE INVENTION

It was surprisingly found now that lactic acid bacteria capable of synthesizing homopolysaccharides, simultaneously also produce large amounts of mannitol. It was also found that such a process of producing mannitol is more cost-effective than the known bacterial processes of producing mannitol, since the sucrose is converted into two valuable components, i.e. mannitol and homopolysaccharides, both of which can be used in several products and processes in the food as well as the non-food industry. Thus, the invention concerns a process of producing mannitol using a bacterium which additionally produces one or more homopolysaccharides and which comprises one or more sucrase activities. Furthermore, the invention concerns the use of such a bacterium in the production of mannitol.

### DESCRIPTION OF THE INVENTION

The present invention pertains to a process of producing mannitol and one or more homopolysaccharides. Preferably, the mannitol and one or more homopolysaccharides are produced simultaneously. Said process comprises the steps of fermenting sucrose by a bacterium expressing mannitol-2-dehydrogenase activity and one or more sucrase activities and recovering the mannitol or homopolysaccharides or both from the medium. These homopolysaccharides comprise fructans, such as inulins or levans, glucans or both. The glucans may e.g. be α-1,6-linked (dextran-type), or α-1,3-linked, or mixed and/or branched α-1,3/1,6-linked (mutan- or alternan-types and the like) or mixed and/or branched α-1,4/1,6-linked (glycogen or amylopectin or high 1,6 types). As an example, the glucans may comprise a structure consisting of terminal, 4substituted, 6-substituted and 4,6-disubstituted α-glucose in a molar ratio of 1.1:2.7:2.5:1.0. Preferably, a bacterium to be applied in the process of the invention uses the fructose units of sucrose for the production of mannitol and uses additionally the glucose units of sucrose for the production of glucans. Alternatively, in the process according to the invention some fructose units can be used for the production of fructans. In the process of the invention, mannitol is produced by virtue of the fact that the bacterium applied in the process expresses D-mannitol-2-dehydrogenase activity. A D-mannitol-2-dehydrogenase is herein defined as an enzyme that catalyses the conversion of D-fructose into D-mannitol, while oxidising NADH to NAD⁺ and/or NADPH to NADP⁺ (E.C. 1.1.1.67 and E. C. 1.1.1.138, respectively).

Sucrases are extracellular enzymes belonging to the group of glycosyl-transferases. Sucrases according to the process of the invention include fructansucrases such as inulosucrases and levansucrases synthesizing inulin and levan, respectively, and glucansucrases synthesizing various glucans from sucrose. Preferably, the bacterium according to the process of the invention comprises one or more glucan-sucrase activities, but it can additionally also comprise one or more fructansucrase activities.

The bacterium to be used in the process according to the invention can be any bacterium capable of expressing mannitol-2-dehydrogenase and additionally capable of expressing one or more sucrase activities. In a preferred embodiment of the process according to the invention the bacterium is a lactic acid bacterium, in particular a lactic acid bacterium selected from the group of genera consisting of Lactobacillus, Leuconostoc and Streptococcus or Lactococcus. Such bacteria are also known in the art, or can be found by analysing lactic acid bacteria strains on the presence of sucrase genes and/or capability of producing homopolysaccharides (especially glucans) from sucrose. Preferred bacteria are strains of Lactobacillus species, such as L. reuteri, L. sake, L. fermentum, L.parabuchneri or related species, or strains of Leuconostoc species such as Lc. mesenteroides, Lc. citreum, or related species.

In a specific embodiment of the process according to the invention the lactic acid bacterium is strain Leuconostoc sp. 86 deposited on 2 May 2001 at the BCCM™/LMG bacteria collection, Belgian Coordinated Collections of Microorganisms (BCCM), Laboratory of Microbiology, Bacteria Collection, University of Gent, K. L. Ledeganck-straat 35, B-9000 Gent, Belgium, under accession number LMG P-20350.

In a preferred specific embodiment of the process according to the invention the lactic acid bacterium is selected from the strains Lactobacillus sp. 33, Lactobacillus reuteri strain 35-5, L. reuteri strain 121, L. reuteri strain 180 and L. reuteri strain 54, deposited on, respectively, 2 May 2001, 8 May 2001, 8 May 2001, 8 May 2001 and 2 May 2001 at the BCCM™/LMG, under accession numbers LMG P-20349, LMG P-18390, LMG P-18388, LMG P-18389 and LMG P-20348, respectively.

In a process according to the invention the fermentation is preferably carried out under (semi-)anaerobic conditions at a temperature of 20-45° C., preferably 32-43° C. and more preferably 35-39° C. and at a sucrose concentration of 10-200 g/l, preferably 50-150 g/l and more preferably 80-120 g/l. Semi-anaerobic conditions in the present invention refer to conditions wherein no oxygen and/or air is supplied during fermentation. The fermentation in the process according to the invention can be operated inter alia in batch mode, fed-batch mode, continuously, semicontinuously and by sucrose feeding.

A process according to the invention also preferably comprises separating mannitol and homopolysaccharides from the fermentation medium by suitable separation and purification techniques known in the art. Preferably, the separation of mannitol and homopolysaccharides is based on the large difference in molecular weight of both. Thus, the two may conveniently be separated by dialysis, ultracentrifugation or size exclusion chromatography. Selective precipitation from solvents such as alcohols or mixtures of water with alcohols or other water-soluble or water-miscible solvents such as acetone, dioxane, tetrahydrofuran and the like, may also be used, optionally i combination with the above-mentioned methods.

In a specific embodiment of the process according to the invention a bacterium is used in which one or more fructansucrase activities have been deleted or reduced. Preferably, said bacterium expresses one or more glucansucrase activities. Bacteria such as for instance lactic acid bacteria may comprise fructansucrase activities producing fructans such as inulins and/or levans from fructose released from sucrose. Deleting or reducing one or more fructansucrase activities may lead to higher mannitol production due to the fact that after such a deleting or reducing step fructose units are more readily available for the enzymes involved in the formation of mannitol. The fructansucrase activities can be deleted or reduced by methods known in the art including, but not limited to, mutagenesis introducing deletions, insertions or substitutions in the DNA encoding enzymes with fructansucrase activity and leading to reduction or deletion of the activity of the fructansucrases, anti-sense RNA techniques, treatment with inhibitors, growing bacteria of interest under conditions conductive to non-expression of enzymes comprising fructansucrase activity or by selection of spontaneous mutants comprising deleted or reduced fructansucrase activities in continuous cultures.

The invention further relates to a bacterium expressing mannitol-2-dehydrogenase activity and one or more sucrase activities for use in the production of mannitol and one or more homopolysaccharides by the fermentation of sucrose. The bacterium is as herein defined above. Preferably, the bacterium is a lactic acid bacterium, in particular a lactic acid bacterium selected from the group of genera consisting of Lactobacillus, Leuconostoc and Streptococcus.

In a preferred embodiment of the invention the lactic acid bacterium according to the invention is strain Leuconostoc sp. 86 deposited at the BCCM™/LMG bacteria collection under accession number LMG P-20350.

In another preferred embodiment of the invention the lactic acid bacterium according to the invention is selected from the strains Lactobacillus sp. strain 33, Lactobacillus reuteri strain 35-5, L. reuteri strain 121, L. reuteri strain 180 and L. reuteri strain 54, deposited at the BCCM™/LMG bacteria collection under accession numbers LMG P-20349, LMG P-18390, LMG P-18388, LMG P-18389 and LMG P-20348, respectively.

In a specific embodiment of a bacterium according to the invention fructan-sucrase activities in the bacterium are deleted or reduced with techniques described above. Preferably, the bacterium expresses one or more glucansucrase activities.

### EXAMPLES

Strains

Lactobacillus sp. 33, deposited on 2 May 2001 as LMG P-20349 in the BCCM™/LMG bacteria collection; Lactobacillus reuteri strain 35-5, deposited on 8 May 2001 as LMG P-18390 in the BCCM™/LMG bacteria collection; Lactobacillus reuteri strain 121, deposited on 8 May 2001 as LMG P-18388 in the BCCM™/LMG bacteria collection; Lactobacillus reuteri strain 180, deposited on 8 May 2001 as LMG P-18389 in the BCCM™/LMG bacteria collection; Lactobacillus reuteri strain 54, deposited on 2 May 2001 as LMG P-20348 in the BCCM™/LMG bacteria collection; Leuconostoc sp. 86 deposited on 2 May 2001 at the BCCM™/LMG bacteria collection under accession number LMG P-20350. All strains were deposited at the Belgian Coordinated Collections of Microorganisms (BCCM), Laboratory of Microbiology, Bacteria Collection, University of Gent, K. L. Ledeganckstraat 35, B-9000 Gent, Belgium

Production Conditions

The strains were grown in MS medium (see de Man et al. (1960) J. Appl. Bacteriol. 23, 130-135) with 100 g/l sucrose (instead of the 20 g/l glucose normally present in this medium) under anaerobic conditions in 1 liter flask at 37° C. After 16-72 hours of growth, the polysaccharides were isolated by precipitation with 2 volumes of cold ethanol. The precipitate was washed with 1 volume of water and the polysaccharides were precipitated again with 2 volumes of cold ethanol and dried.

The manitol produced in the supernatants of the cultures was analysed by an BPLC-system using a cation-exchange column (sulfonated styrene-divinylbenzene copolymer) The temperature of the column was 85° C. and 100 ppm Ca-EDTA was used as an eluent at an elution rate of 0.4 ml/min and RI detection.

Results

TABLE 1
Production of mannitol and polysaccharides
from several lactic acid bacteria strains.

| | Amount of mannitol | Amount of polysaccharide |
|---|---|---|
| strain | produced (g/l) | produced (g/l) |
| Lactobacillus sp. 33 | 34 | >30 |
| Lactobacillus reuteri | 30 | 23 |
| strain 35-5 | | |
| Lactobacillus reuteri | 30 | 11 |
| strain 121 | | |
| Lactobacillus reuteri | 26 | 19 |
| strain 180 | | |
| Lactobacillus reuteri | >34 | 4 |
| strain 54 | | |
| Leuconostoc sp. 86 | 22 | 20 |

1-11. canceled

12. A process of producing mannitol and/or one or more homopolysaccharides, the process comprising the steps of : a) fermenting sucrose by a bacterium expressing mannitol-2-dehydrogenase activity and one or more sucrase activities, b) recovering the mannitol or homopolysaccharides or both from the medium.

13. A process according to claim 12, wherein the bacterium is a lactic acid bacterium.

14. A process according to claim 12, wherein the bacterium is selected from the genera Lactobacillus, Leuconostoc and Streptococcus.

15. A process according to claim 14, wherein the bacterium is selected from the strains Lactobacillus sp. 33, Lactobacillus reuteri strain 35-5, Lactobacillus reuteri strain 121, Lactobacillus reuteri strain 180, Lactobacillus reuteri strain 54 and Leuconostoc sp. 86, deposited at the BCCM/LMG bacteria collection under accession numbers LMG P-20349, LMG P-18390, LMG P-18388, LMG P-18389, LMG P-20348 and LMG P-20350, respectively.

16. A process according to claim 12, wherein the fermentation is carried out under (semi-) anaerobic conditions at a temperature of 20-45 C at a sucrose concentration of 10-200 g/l.

17. A process according to claim 16, wherein the fermentation is carried out at a temperature of 32-43° C. and./or at a sucrose concentration of 50-150 g/l.

18. A process according to claim 12, wherein a bacterium is used in which one or more frutansucrase activities have been deleted or reduced and wherein the bacterium express one or more glucansucrase activities.
